(19) United States
(12) Patent Application Publication
(10) Pub. No.: US 2026/0137693 A1
(43) Pub. Date: May 21, 2026

(54) CANCER TREATMENTS USING MTA-COOPERATIVE PRMT5 INHIBITORS AND MAT2A INHIBITORS

(71) Applicants: AMGEN INC., Thousand Oaks, CA (US); Ideaya Biosciences, Inc., South San Francisco, CA (US)

(72) Inventors: Brian Belmontes, Thousand Oaks, CA (US); Paul E. Hughes, Ventura, CA (US); Claire L. Neilan, South San Francisco, CA (US); Katherine Slemmons, Los Angeles, CA (US); Michael A. White, South San Francisco, CA (US); Marcus M. Fischer, South San Francisco, CA (US)

(21) Appl. No.: 19/132,928

(22) PCT Filed: Nov. 30, 2023
(86) PCT No.: PCT/US2023/081789

§ 371 Date: May 27, 2025

Related U.S. Application Data
(60) Provisional application No. 63/428,911, filed on Nov. 30, 2022.
(60) Provisional application No. 63/448,396, filed on Feb. 27, 2023.
(60) Provisional application No. 63/458,501, filed on Apr. 11, 2023.

(51) Int. Cl. A61K 31/5377; A61K 31/519; A61P 35/00

(52) U.S. Cl. CPC A61K 31/5377; A61K 31/519; A61P 35/00

(57) ABSTRACT

Described herein are methods of treating cancer in a patient comprising administering a combination therapy of a therapeutically effective amount of a PRMT5 inhibitor (Compound A) and a therapeutically effective amount of a MAT2A inhibitor (Compound B) to the patient.

### BACKGROUND

Epigenetic regulation of gene expression is an important biological determinant of protein production and cellular differentiation and plays a significant pathogenic role in a number of human diseases. Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease. PRMT5 plays a role in diseases such as proliferative disorders, metabolic disorders, and blood disorders.

The homozygous deletion of tumor suppressor genes is a key driver of cancer, frequently resulting in the collateral loss of passenger genes located in close genomic proximity to the tumor suppressor. Deletion of these passenger genes can create therapeutically tractable vulnerabilities that are specific to tumor cells. Homozygous deletion of the chromosome 9p21 locus, which harbors the well-known tumor suppressor CDKN2A (cyclin dependent kinase inhibitor 2A), occurs in 15% of all tumors and frequently includes the passenger gene MTAP (methylthioadenosine phosphorylase), a key enzyme in the methionine and adenine salvage pathways. Deletion of MTAP results in accumulation of its substrate, methylthioadenosine (MTA). MTA shares close structural similarity to S-adenosylmethionine (SAM), the substrate methyl donor for the type II methyltransferase PRMT5. Elevated MTA levels, driven by loss of MTAP, selectively compete with SAM for binding to PRMT5, placing the methyltransferase in a hypomorphic state, vulnerable to further PRMT5 inhibition. Multiple genome scale shRNA drop out screens performed in large tumor cell line panels have identified a strong correlation between MTAP loss and cell line dependency on PRMT5, further highlighting the strength of this metabolic vulnerability. However, PRMT5 is a known cell essential gene and conditional PRMT5 knockout and siRNA knockdown studies suggest that significant liabilities could be associated with inhibiting PRMT5 in normal tissues (e.g. pan-cytopenia, infertility, skeletal muscle loss, cardiac hypertrophy, others). Therefore, novel strategies are required to exploit this metabolic vulnerability and preferentially target PRMT5 in MTAP null tumors while sparing PRMT5 in normal tissues (MTAP WT). Targeting PRMT5 with an MTA-cooperative small molecule inhibitor could preferentially target the MTA bound state of PRMT5, enriched in MTAP null tumor cells, while providing an improved therapeutic index over normal cells where MTAP is intact and MTA levels are low.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA, and proteins. One methylase that utilizes SAM as a methyl donor is PRMT5. While SAM is required for PRMT5 activity, PRMT5 is competitively inhibited by MTA. Since MTA is part of the methionine salvage pathway, cellular MTA levels stay low in a process initiated by MTAP.

MTAP is in a locus on chromosome 9 that is often deleted in cells of patients with cancers from several tissues of origin including central nervous system, pancreas, esophageal, bladder and lung. Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted (also referred to herein as MTAP-null) cells compare to MTAP WT cells (McDonald et. al. 2017 Cell 170, 577-592). Furthermore, inducible knockdown of MAT2A protein decreased tumor growth in vivo (Maqon et. al., 2016 Cell Reports 15(3), 574-587).

### SUMMARY

The disclosure provides methods of treating cancer in a patient in need thereof comprising administering a combination therapy of a therapeutically effective amount of Compound A or pharmaceutically acceptable salt thereof and a therapeutically effective amount of Compound B or pharmaceutically acceptable salt thereof to the patient. In various cases, the patient suffers from a MTAP-deleted cancer. In some cases, the cancer is a solid tumor. The disclosure also provides a combination product comprising a therapeutically effective amount of Compound A or pharmaceutically acceptable salt thereof and a therapeutically effective amount of Compound B or pharmaceutically acceptable salt thereof. The combination product is useful for the treatment of a variety of cancers, including solid tumors.

### BRIEF DESCRIPTION OF THE FIGURES

FIG. 1 is a dose response curve showing the effect of the combination of Compound A and Compound B in a NSCLC cell line (CALU1).

FIG. 2 is a dose response curve showing the effect of the combination of Compound A and Compound B in a pancreatic cancer cell line (BxPC3).

FIG. 3 is a graph showing that the combination of Compound A and Compound B resulted in significant anti-tumor activity versus either single agent alone in BxPC-3 pancreatic carcinoma xenografts.

FIGS. 4A and 4B are graphs showing that the combination of Compound A and Compound B resulted in significant anti-tumor activity versus either single agent alone in H838 NSCLC xenografts.

FIG. 5 is a graph showing that the combination of Compound A and Compound B resulted in significant anti-tumor activity versus either single agent alone that was maintained following cessation of dosing in H838 NSCLC xenografts.

### DETAILED DESCRIPTION

The disclosure provides methods of treating cancer in a patient in need thereof comprising administering to the patient a combination therapy of a therapeutically effective amount of Compound A or pharmaceutically acceptable salt thereof and a therapeutically effective amount of Compound B or pharmaceutically acceptable salt thereof to the patient. A “patient” or “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “patient” and “subject” are used interchangeably herein.

The disclosure also provides a combination product comprising a therapeutically amount of Compound A or pharmaceutically acceptable salt thereof and a therapeutically effective amount of Compound B or pharmaceutically acceptable salt thereof. The combination product is useful for the treatment of a variety of cancers, including solid tumors. The term “combination product” means in which each therapeutic agent in the combination is individually formulated into its own pharmaceutical composition and each of the pharmaceutical compositions are administered in the same medical treatment (for example, the same medical treatment of cancer). In some embodiments, each of the pharmaceutical compositions may have the same or different carriers, diluents or excipients. Also provided herein is use of a therapeutically effective amount of Compound A or pharmaceutically acceptable salt thereof and a therapeutically effective amount of Compound B or pharmaceutically acceptable salt thereof in treating cancer. Provided herein is use of a therapeutically effective amount of Compound A or pharmaceutically acceptable salt thereof and a therapeutically effective amount of Compound B or pharmaceutically acceptable salt thereof in the manufacture of a medicament for treating cancer.

Compound A is a PRMT5 inhibitor that has a chemical name (S)-(4-amino-1,3-dihydrofuro[3,4-c][1,7]naphthyridin-8-yl)(3-(4-(trifluoromethyl)phenyl)morpholino)methanone and has a structure of Compound A:

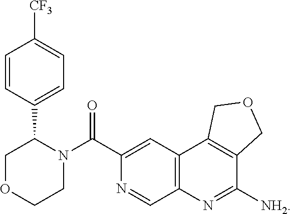 [SMILES: Nc1nc2cnc(C(=O)N3CCOC[C@@H]3c3ccc(C(F)(F)F)cc3)cc2c2c1COC2]

Compound A can be synthesized using methods as described, e.g., in PCT/US22/75648 and PCT/US21/63540.

In the methods disclosed herein, Compound A can be administered as a pharmaceutically acceptable salt. Pharmaceutically acceptable salts include those derived from suitable inorganic and organic acids and bases. Pharmaceutically acceptable salts include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Suitable salts include those described in P. Heinrich Stahl, Camille G. Wermuth (Eds.), Handbook of Pharmaceutical Salts Properties, Selection and Use; 2002. In various embodiments, Compound A or salt thereof is administered orally.

A “therapeutically effective amount” of Compound A means an amount effective to treat or to prevent development of, or to alleviate the existing symptoms of, the patient being treated. Determination of the effective amounts is well within the capability of those skilled in the art, especially in light of the detailed disclosure provided herein. Generally, a “therapeutically effective dose” refers to that amount of a Compound A described herein that results in achieving the desired effect. For example, a therapeutically effective amount of Compound A described herein decreases PRMT5 activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

Compound B is a MAT2A inhibitor that has a chemical name 4-amino-1-(2-chlorophenyl)-7-(trifluoromethyl)pyrido[2,3-d]pyrimidin-2(1H)-one and has the following structure.

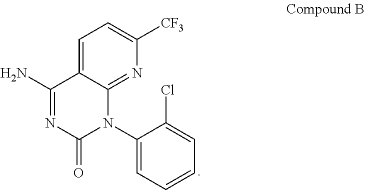 [SMILES: Nc1nc(=O)n(-c2ccccc2Cl)c2nc(C(F)(F)F)ccc12]

Compound B, and methods of making Compound B are disclosed in PCT/US19/65260 (WO 2020/123395).

In the methods disclosed herein, Compound B can be administered as a pharmaceutically acceptable salt. Pharmaceutically acceptable salts include those derived from suitable inorganic and organic acids and bases. Pharmaceutically acceptable salts include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Suitable salts include those described in P. Heinrich Stahl, Camille G. Wermuth (Eds.), Handbook of Pharmaceutical Salts Properties, Selection and Use; 2002. In various embodiments, Compound B or salt thereof is administered orally.

A “therapeutically effective amount” of Compound B means an amount effective to treat or to prevent development of, or to alleviate the existing symptoms of, the patient being treated. Determination of the effective amounts is well within the capability of those skilled in the art, especially in light of the detailed disclosure provided herein. Generally, a “therapeutically effective dose” refers to that amount of a Compound B described herein that results in achieving the desired effect. For example, a therapeutically effective amount of Compound A described herein decreases MAT2A activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

### Cancer

In some embodiments, the cancer is a MTAP-deleted cancer. A MTAP-deleted (or “MTAP-null”) cancer refers to a cancer that lacks expression of the enzyme methylthioadenosine phosphorylase (MTAP). The MTAP gene, located at chromosomal locus 9p21 is frequently co-deleted with the CDKN2A and CDKN2B genes. Selective MTAP deficiency, refers to deficiency without co-deletion of the CDKN2 genes, due either to selective deletion of the MTAP locus or to methylation of the MTAP promoter. MTAP-null cancers include MTAP-deficiency in at least 1% of disease cells. Terms “MTAP-null” and “MTAP-deleted” are used interchangeably herein.

In some embodiments, the cancer is an MTAP-deficient and/or MTA-accumulating cancer. An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In some embodiments, the cancer is a MTAP-deleted cancer. In a patient afflicted with a MTAP-deficiency-related disease, it is possible that some disease cells (e.g., cancer cells) can be MTAP-deficient while others are not. Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of Compound A or a pharmaceutically acceptable salt thereof and administration of Compound B or a pharmaceutically acceptable salt thereof. Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

In some embodiments, the cancer is cancer such as acute myeloid leukemia, cancer in adolescents, adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some cases, the cancer is pancreatic cancer; esophageal cancer; melanoma; lung cancer; mixed mullerian cancer; ovarian cancer; or gallbladder cancer.

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the cancer is leukemia, glioma, melanoma, pancreatic, non-small cell lung cancer (NSCLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma or mesothelioma. In various embodiments, the cancer is bladder cancer, melanoma, brain cancer, lung cancer, pancreatic cancer, breast cancer, esophageal cancer, head and neck cancer, kidney cancer, colon cancer, diffuse large B cell lymphoma (DLBCL), acute lymphoblastic leukemia (ALL) or mantle cell lymphoma (MCL). In some embodiments, the cancer is gastric cancer. In some embodiments, the cancer is colon cancer. In some embodiments, the cancer is liver cancer. In some embodiments, the cancer is glioblastoma multiforme (GBM). In some embodiments, the cancer is bladder cancer. In various embodiments, the cancer is esophageal cancer. In some embodiments, the cancer is breast cancer. In some embodiments, the cancer is NSCLC. In some embodiments, the cancer is MCL. In some embodiments, the cancer is DLBCL. In various embodiments, the cancer is ALL. In some embodiments, the cancer is lung cancer or pancreatic cancer.

In some embodiments, the MTAP-null cancer is lung cancer, biliary tract cancer, head and neck squamous cell carcinoma, pancreatic adenocarcinoma, gallbladder cancer, or mesothelioma.

In some embodiments, the cancer is a solid tumor. In some embodiments, the tumor is malignant.

Exemplary MTAP-null solid tumors include, but are not limited to, MTAP-null brain cancer (including, but not limited to, MTAP-null glioma, MTAP-null oligodendroglioma, MTAP-null glioblastoma multiforme, MTAP-null astrocytoma, MTAP-null medulloblastoma, MTAP-null ependymoma, and MTAP-null meningioma), MTAP-null head and neck cancer (including, but not limited to, MTAP-null salivary gland (parotid) tumors, MTAP-null head and neck squamous cell carcinoma, and MTAP-null thyroid cancer), MTAP-null breast cancer (including, but not limited to, invasive ductal breast cancer, mixed mucinous breast cancer and lobular carcinoma), MTAP-null mesothelioma, MTAP-null gastrointestinal tract cancer (including but not limited to, MTAP-null esophageal cancer (including, but not limited to, adenocarcinoma and squamous cell carcinoma), MTAP-null gastro-esophageal junction cancer, MTAP-null stomach cancer (including, but not limited to, adenocarcinoma and signet ring cell carcinoma), MTAP-null small bowel cancer, MTAP-null colon cancer, MTAP-null rectal cancer and MTAP-null gastrointestinal stromal tumor), MTAP-null neuroendocrine tumor, MTAP-null hepatobiliary cancer (including, but not limited to, MTAP-null biliary tract cancer (including cholangiocarcinoma, gallbladder cancer and ampullary cancer) and MTAP-null hepatocellular carcinoma), MTAP-null pancreatic cancer (including pancreatic adenocarcinoma), MTAP-null kidney cancer (including, but not limited to, MTAP-null renal cell carcinoma), MTAP-null adrenocortical carcinoma, MTAP-null bladder cancer (including, but not limited to, MTAP-null urothelial carcinoma), MTAP-null adrenocortical carcinoma, MTAP-null endometrial cancer, MTAP-null uterine cancer, MTAP-null testicular cancer, MTAP-null germ cell tumor, or MTAP-null prostate cancer, MTAP-null sarcoma or MTAP-null bone cancer (including, but not limited to, MTAP-null osteosarcoma, MTAP-null chondrosarcoma, MTAP-null soft tissue sarcoma, MTAP-null Ewing sarcoma, MTAP-null liposarcoma, MTAP-null leiomyosarcoma, and MTAP-null myxofibrosarcoma), MTAP-null cutaneous tumors (MTAP-null cutaneous squamous cell carcinoma and MTAP-null melanoma), MTAP-null nerve sheath tumor and MTAP-null cancer of unknown primary (CUP).

In some embodiments, the MTAP-null cancer is a hematologic tumor. Exemplary hematologic tumors include, but are not limited to, MTAP-null leukemia (including, but not limited to, MTAP-null acute lymphocytic leukemia, MTAP-null acute myeloid leukemia), MTAP-null lymphoma (including, but not limited to, MTAP-null mantle cell lymphoma, MTAP-null follicular lymphoma, MTAP-null diffuse large B cell lymphoma, and MTAP-null mycosis fungoides).

### Monitoring Efficacy of Treatment

The efficacy of a given treatment for cancer can be determined by the skilled clinician. However, a treatment is considered “effective treatment,” as the term is used herein, if any one or all of the signs or symptoms of e.g., a tumor are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

In some embodiments, the combination therapy described herein exhibits a combination benefit. The term “combination benefit” refers to an observed efficacy with a combination therapy that is higher than treatment with either individual therapy alone. In some embodiments, the combination therapy described herein exhibits a combination benefit compared to Compound A monotherapy. In some embodiments, the combination therapy described herein exhibits a combination benefit compared to Compound B monotherapy.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention, except for any definitions, subject matter disclaimers or disavowals, and except to the extent that the incorporated material is inconsistent with the express disclosure herein, in which case the language in this disclosure controls. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Exemplary Embodiments

1. A method of treating cancer in a patient in need thereof comprising administering a combination therapy of a therapeutically effective amount of Compound A or pharmaceutically acceptable salt thereof and a therapeutically effective amount of Compound B or pharmaceutically acceptable salt thereof to the patient.

2. The method of embodiment 1, wherein the cancer is a MTAP-deleted cancer.

3. The method of embodiment 1 or 2, wherein the cancer is a solid tumor.

4. The method of embodiment one of claims 1-3, wherein the cancer is lung cancer.

5. The method of any one of embodiments 1-3, wherein the cancer is pancreatic cancer.

6. The method of any one of embodiments 1-5, wherein Compound A or salt thereof is administered orally.

7. The method of any one of embodiments 1-6, wherein Compound A or salt thereof is administered once daily.

8. The method of any one of embodiments 1-7, wherein Compound B or salt thereof is administered orally.

9. The method of any one of embodiments 1-8, wherein Compound A and Compound B are administered concurrently.

10. The method of any one of embodiments 1-8, wherein Compound A and Compound B are administered sequentially.

11. The method of any one of embodiments 1-10, wherein the therapeutically effective amount of Compound A or salt thereof is less than an amount of Compound A or salt thereof as a monotherapy.

12. The method of any one of embodiment 1-11, wherein the therapeutically effective amount of Compound B or salt thereof is less than an amount of Compound B or salt thereof as a monotherapy.

13. The method of any one of embodiments 1-12, wherein the combination therapy exhibits combination benefit compared to Compound A monotherapy.

14. The method of any one of embodiments 1-13, wherein the combination therapy exhibits combination benefit compared to Compound B monotherapy.

15. A combination product comprising a therapeutically effective amount of Compound A or pharmaceutically acceptable salt thereof and a therapeutically effective amount of Compound B or pharmaceutically acceptable salt thereof.

16. Use of a therapeutically effective amount of Compound A or pharmaceutically acceptable salt thereof and a therapeutically effective amount of Compound B or pharmaceutically acceptable salt thereof in treating cancer.

17. Use of a therapeutically effective amount of Compound A or pharmaceutically acceptable salt thereof and a therapeutically effective amount of Compound B or pharmaceutically acceptable salt thereof in the manufacture of a medicament for treating cancer.

18. The use of embodiment 16 or 17, wherein the therapeutically effective amount of Compound A or pharmaceutically acceptable salt thereof and a therapeutically effective amount of Compound B or pharmaceutically acceptable salt thereof are administered concurrently.

19. The use of embodiment 17 or 18, wherein the therapeutically effective amount of Compound A or pharmaceutically acceptable salt thereof and a therapeutically effective amount of Compound B or pharmaceutically acceptable salt thereof are administered sequentially.

### Additional Exemplary Embodiments

1. A method of treating cancer in a patient in need thereof comprising administering a combination therapy of a therapeutically effective amount of Compound A or pharmaceutically acceptable salt thereof and a therapeutically effective amount of Compound B or pharmaceutically acceptable salt thereof to the patient.

2. The method of embodiment 1, wherein the cancer is a MTAP-deleted cancer.

3. The method of embodiment 1, wherein the cancer is an MTAP-deficient cancer, MTA-accumulating cancer, or a combination thereof.

4. The method of embodiment 1 or 2, wherein the cancer is a solid tumor.

5. The method of embodiment 4, wherein the tumor is malignant.

6. The method of any one of embodiments 1-4, wherein the cancer is lung cancer.

7. The method of any one of embodiments 1-4, wherein the cancer is pancreatic cancer.

8. The method of embodiment 2, wherein the MTAP-deleted cancer is lung cancer.

9. The method of embodiment 8, wherein the lung cancer is non-squamous cell lung cancer (NSCLC).

10. The method of embodiment 2, wherein the MTAP-deleted cancer is biliary tract cancer.

11. The method of embodiment 2, wherein the MTAP-deleted cancer is head and neck squamous cell carcinoma.

12. The method of embodiment 2, wherein the MTAP-deleted cancer is pancreatic adenocarcinoma.

13. The method of embodiment 2, wherein the MTAP-deleted cancer is gallbladder cancer.

14. The method of embodiment 2, wherein the MTAP-deleted cancer is mesothelioma.

15. The method of embodiment 2, wherein the cancer is not a primary brain tumor or lymphoma.

16. The method of any one of embodiments 1-15, wherein Compound A or salt thereof is administered orally.

17. The method of any one of embodiments 1-16 wherein Compound A or salt thereof is administered once daily.

18. The method of any one of embodiments 1-17, wherein Compound B or salt thereof is administered orally.

19. The method of any one of embodiments 1-18, wherein Compound A or salt thereof and Compound B or salt thereof are administered concurrently.

20. The method of any one of embodiments 1-18, wherein Compound A or salt thereof and Compound B or salt thereof are administered sequentially.

21. The method of any one of embodiments 1-20, wherein the therapeutically effective amount of Compound A or salt thereof is less than an amount of Compound A or salt thereof as a monotherapy.

22. The method of any one of embodiments 1-21, wherein the therapeutically effective amount of Compound B or salt thereof is less than an amount of Compound B or salt thereof as a monotherapy.

23. The method of any one of embodiments 1-22, wherein the combination therapy exhibits combination benefit compared to Compound A or salt thereof monotherapy.

24. The method of any one of embodiments 1-23, wherein the combination therapy exhibits combination benefit compared to Compound B or salt thereof monotherapy.

25. The method of any one of embodiments 1-24, wherein the cancer is deficient in CDKN2A.

26. A combination product comprising a therapeutically effective amount of Compound A or pharmaceutically acceptable salt thereof and a therapeutically effective amount of Compound B or pharmaceutically acceptable salt thereof.

27. Use of a therapeutically effective amount of Compound A or pharmaceutically acceptable salt thereof and a therapeutically effective amount of Compound B or pharmaceutically acceptable salt thereof in treating cancer.

28. Use of a therapeutically effective amount of Compound A or pharmaceutically acceptable salt thereof and a therapeutically effective amount of Compound B or pharmaceutically acceptable salt thereof in the manufacture of a medicament for treating cancer.

29. The use of embodiment 27 or 28, wherein the cancer is a MTAP-deleted cancer.

30. The use of embodiment 27 or 28, wherein the cancer is an MTAP-deficient cancer, MTA-accumulating cancer, or a combination thereof.

31. The use of embodiment 29 or 30, wherein the cancer is a solid tumor.

32. The use of embodiment 31, wherein the tumor is malignant.

33. The use of any one of embodiments 27-32, wherein the cancer is lung cancer.

34. The use of any one of embodiments 27-32, wherein the cancer is pancreatic cancer.

35. The use of embodiment 29, wherein the MTAP-deleted cancer is lung cancer.

36. The use of embodiment 35, wherein the lung cancer is non-squamous cell lung cancer (NSCLC).

37. The use of embodiment 29, wherein the MTAP-deleted cancer is biliary tract cancer.

38. The use of embodiment 29, wherein the MTAP-deleted cancer is head and neck squamous cell carcinoma.

39. The use of embodiment 29, wherein the MTAP-deleted cancer is pancreatic adenocarcinoma.

40. The use of embodiment 29, wherein the MTAP-deleted cancer is gallbladder cancer.

41. The use of embodiment 29, wherein the MTAP-deleted cancer is mesothelioma.

42. The use of embodiment 29, wherein the cancer is not a primary brain tumor or lymphoma.

43. The use of any one of embodiments 27-42, wherein Compound A or salt thereof is administered orally.

44. The use of any one of embodiments 27-43 wherein Compound A or salt thereof is administered once daily.

45. The use of any one of embodiments 27-44, wherein Compound B or salt thereof is administered orally.

46. The use of any one of embodiments 27-45, wherein Compound A or salt thereof and Compound B or salt thereof are administered concurrently.

47. The use of any one of embodiments 27-45, wherein Compound A or salt thereof and Compound B or salt thereof are administered sequentially.

48. The use of any one of embodiments 27-47, wherein the therapeutically effective amount of Compound A or salt thereof is less than an amount of Compound A or salt thereof as a monotherapy.

49. The use of any one of embodiments 27-48, wherein the therapeutically effective amount of Compound B or salt thereof is less than an amount of Compound B or salt thereof as a monotherapy.

50. The use of any one of embodiments 27-49, wherein the combination therapy exhibits combination benefit compared to Compound A or salt thereof monotherapy.

51. The use of any one of embodiments 27-50, wherein the combination therapy exhibits combination benefit compared to Compound B or salt thereof monotherapy.

52. The use of any one of embodiments 27-51, wherein the cancer is deficient in CDKN2A.

### EXAMPLES

### Example 1—Combination of Compound A and Compound B in Lung Cancer Cell Lines

Lung cancer cell lines (LU99 and H838) were treated with the combination of Compound A and Compound B for 6 days. Compound A was performed at a 1.9-fold dilution series and Compound B was performed at 1.9-dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{Treatment}{Average of vehicle} )$$

Synergy analysis was performed using the CalcuSyn software to determine Combination Index (CI) scores based on the drug concentrations used and corresponding Fa values. Results are shown in Tables 1-2 below. *CI Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1. Tables 1 and 2 represent 1 of the 2 studies conducted. N=2 technical replicates.

TABLE 1
Representative Compound A and Compound B concentrations and
corresponding combination Fa and CI scores in LU99 cells.
LU99 Combination Index (CI) Determination

| Dose, Fixed | Dose, Variable | Combination | Computed |
|---|---|---|---|
| Compound A (μM) | Compound B (μM) | Fa | CI value* |
| 0.040 | 1 | 0.662 | 0.241 |
| 0.040 | 0.526 | 0.661 | 0.182 |
| 0.040 | 0.277 | 0.636 | 0.181 |
| 0.040 | 0.146 | 0.599 | 0.203 |
| 0.040 | 0.077 | 0.631 | 0.149 |
| 0.040 | 0.040 | 0.615 | 0.156 |
| 0.040 | 0.021 | 0.606 | 0.159 |

TABLE 2
Representative Compound A and Compound B concentrations and
corresponding combination Fa and CI scores in H838 cells.
H838 Combination Index (CI) Determination

| Dose, Fixed | Dose, Variable | Combination | Computed |
|---|---|---|---|
| Compound A (μM) | Compound B (μM) | Fa | CI value* |
| 0.146 | 0.2 | 0.952 | 0.245 |
| 0.146 | 0.105 | 0.932 | 0.327 |
| 0.146 | 0.055 | 0.917 | 0.362 |
| 0.146 | 0.029 | 0.916 | 0.328 |
| 0.146 | 0.015 | 0.905 | 0.351 |
| 0.146 | 0.008 | 0.872 | 0.467 |
| 0.146 | 0.004 | 0.863 | 0.485 |

### Example 2—Compound A and Compound B in Lung Cancer and Pancreatic Cell Lines

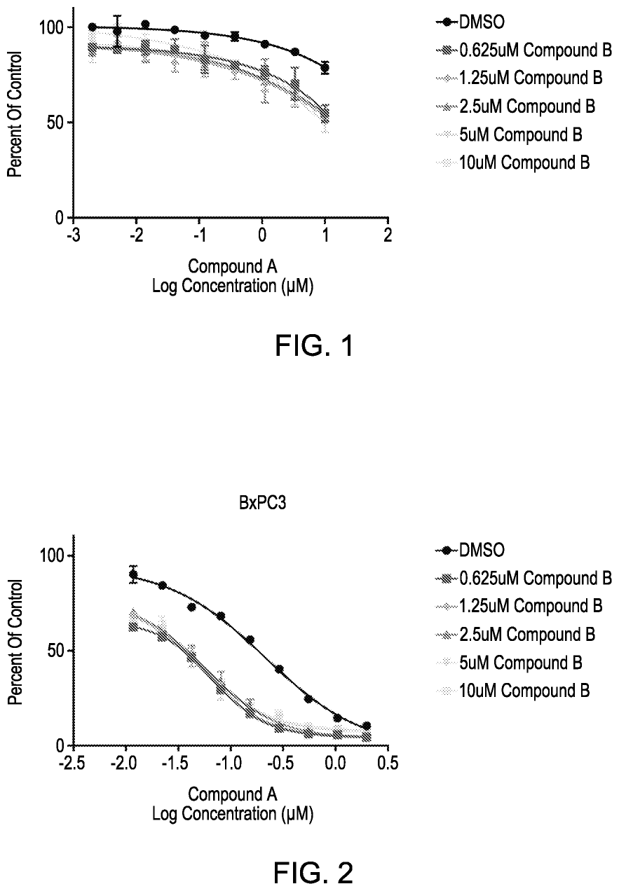

NSCLC cancer cell line CALU1 was treated with the combination of Compound A and Compound B for 6 days. Top concentration of Compound A tested was 10 μM with a total of nine 1.9-fold dilutions and DMSO-only control. Top concentration of Compound B tested was 10 μM with a total of five 2-fold dilutions and DMSO-only control. Cell viability was measured by the CellTiter-Glo Luminescence assay. Raw luminescent values were converted to POC with the following equation: POC=100*(Treatment/Vehicle). FIG. 1 represents 1 of the 2 studies conducted. N=2 technical replicates. Mean and standard deviation graphed.

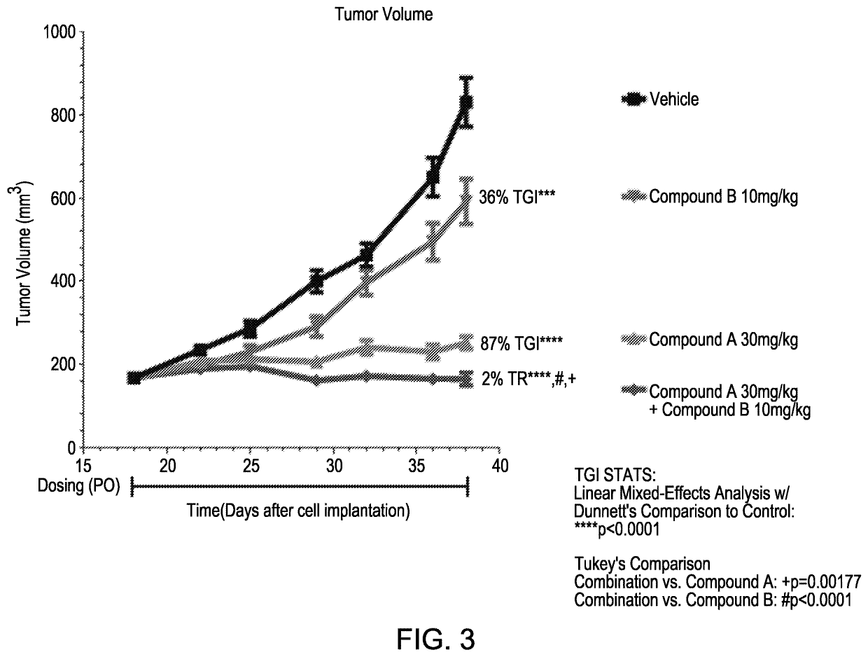

Pancreatic cancer cell line BxPC3 was treated with the combination of Compound A and Compound B for 6 days. Top concentration of Compound A tested was 2 μM with a total of nine 1.9-fold dilutions and DMSO-only control. Top concentration of Compound B tested was 10 μM with a total of five 2-fold dilutions and DMSO-only control. Cell viability was measured by the CellTiter-Glo Luminescence assay. Raw luminescent values were converted to POC with the following equation: POC=100*(Treatment/Vehicle). FIG. 2 represents 1 of the 3 studies conducted. N=2 technical replicates. Mean and standard deviation graphed.

Combination benefit is observed in cancer cell line models that exhibit sensitivity to both single agents, as shown in Example 1). In the context of models that are insensitive to Compound B as a single agent, combination benefit cannot be assessed, and no dose-dependent combination effects are observed. However, there is a greater decrease in viability with the combination suggesting there may be an additive benefit of the combination in these models (as shown in Example. 2).

### Example 3—Compound A and Compound B Show Combination Benefit in a Pancreatic Cancer Animal Model

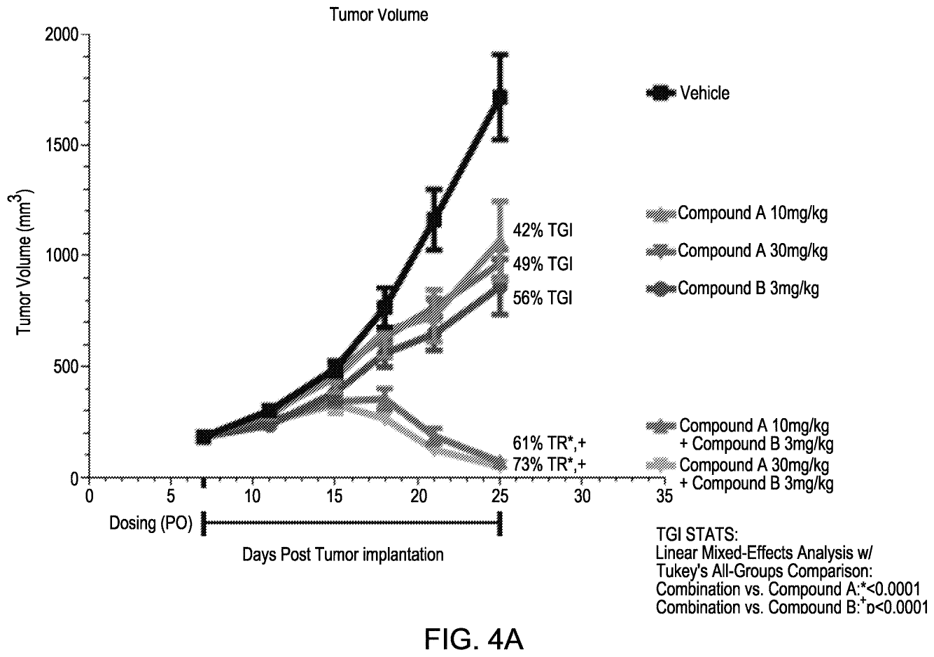

BxPC-3 cells (5×10⁶) were re-suspended in 100 μL of serum free media with 1:1 Matrigel and injected subcutaneously in the right flank of CB.17 SCID mice. Animals were sorted based on tumor volumes on day 18. Animals were orally dosed once daily with either vehicle (0.1% Tween®80, 2% HPMC in DI water, pH 2), Compound A at 30 mg/kg, or Compound B at 10 mg/kg. Tumor volumes were measured twice a week with digital calipers. Plotted data represents group means±SEM for each group. N=10 for each group. Results show that a combination of Compound A and Compound B resulted in significant anti-tumor activity versus either single agent alone in BxPC-3 pancreatic carcinoma xenografts (see FIG. 3).

### Example 4—The Effect of Compound A in Combination with Compound B on Tumor Growth in the H838 NSCLC Xenograft Model in Female Athymic Nude Mice

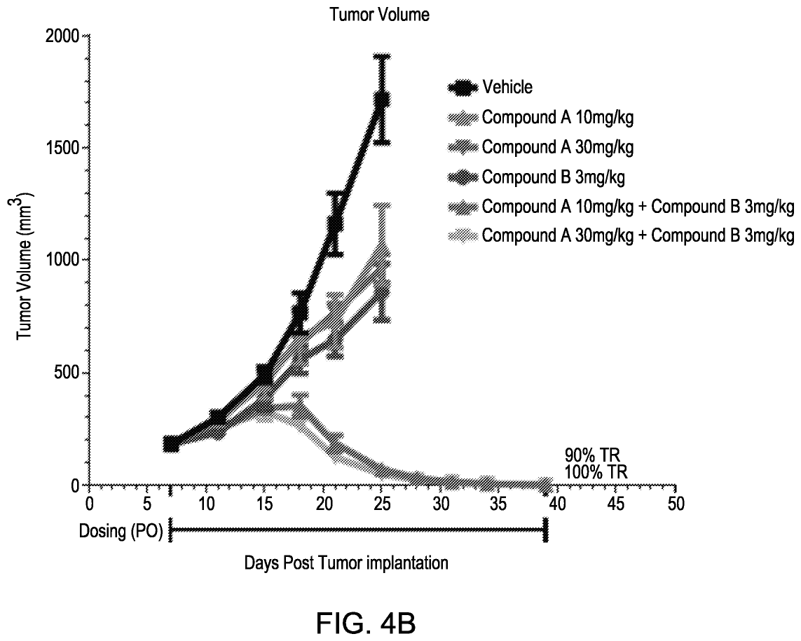

On day 7, female Athymic nude mice were sorted into six groups (n=10) with mean tumor volumes of 185 mm³ and dosing was initiated. Vehicle 1 (0.1% Tween®80, 2% HPMC in DI water, pH 2), vehicle 2 (0.5% methylcellulose (400 cps) 0.5% Tween 80 without pH adjustment) and test agents were administered orally (PO) once a day as follows: Group 1 received vehicle 1 and vehicle 2. Group 2 received 10 mg/kg Compound A and vehicle 2; Group 3 received 30 mg/kg Compound A and vehicle 2; Group 4 received 3 mg/kg Compound B and vehicle 1; Group 5 received 10 mg/kg Compound A and 3 mg/kg Compound B; Group 6 received 30 mg/kg Compound A and 3 mg/kg Compound B. Tumor volumes were monitored twice a week with digital calipers. Data represent group mean±SEM, n=10 for each group. Treatments and tumor measurement for Group 1 to group 4 were stopped on Day 25. Treatments for Group 5 and Group 6 stopped on Day 39. Results show that a combination of Compound A and Compound B resulted in significant anti-tumor activity versus either single agent alone in H838 NSCLC xenografts (see FIG. 4A).

The experiment was repeated and again results show that a combination of Compound A and Compound B resulted in significant anti-tumor activity versus either single agent alone in H838 NSCLC xenografts (see FIG. 4B).

### Example 5—Combination of Compound A and Compound B in Lung Cancer Cell Lines

Lung cancer cell lines (H838) were treated with the combination of Compound A and Compound B for 6 days. Compound A was performed at a 1.9-fold dilution series and Compound B was performed at 1.9-dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{Treatment}{Average of vehicle} )$$

Synergy analysis was performed using the CalcuSyn software to determine Combination Index (CI) scores based on the drug concentrations used and corresponding Fa values. Results are shown in Table 3 below. *CI Values (Calcusyn): C<1 indicates synergy; C=1 indicates additivity; C>1 indicates antagonism.

TABLE 3
Representative Compound A and Compound B concentrations
and corresponding combination CI scores in H838 cells.

| | | | | | | | | | | Compound B |
|---|---|---|---|---|---|---|---|---|---|---|
| | | | | | | | | | | (μM) |
| | 1.01 | 0.54 | 0.35 | 0.25 | 0.28 | 0.46 | 0.68 | 1.17 | 1.66 | 0.200 |
| | 1.03 | 0.65 | 0.47 | 0.33 | 0.37 | 0.52 | 0.65 | 0.97 | 1.01 | 0.105 |
| | 1.30 | 0.78 | 0.48 | 0.36 | 0.36 | 0.57 | 0.73 | 0.88 | 1.15 | 0.055 |
| | 1.24 | 0.70 | 0.43 | 0.33 | 0.37 | 0.48 | 0.55 | 0.80 | 0.78 | 0.029 |
| | 1.19 | 0.75 | 0.45 | 0.35 | 0.41 | 0.49 | 0.68 | 0.98 | 0.62 | 0.p15 |
| | 1.23 | 0.79 | 0.54 | 0.47 | 0.49 | 0.70 | 0.82 | 1.20 | 1.21 | 0.008 |
| | 1.21 | 0.87 | 0.56 | 0.49 | 0.67 | 0.98 | 1.51 | 1.56 | 2.86 | 0.004 |
| Compound A | 1 | 0.526 | 0.277 | 0.146 | 0.077 | 0.040 | 0.021 | 0.011 | 0.006 | |
| (μM) | | | | | | | | | | |

### Example 6—Compound a and Compound B Show Combination Benefit in a Lung Cancer Animal Model

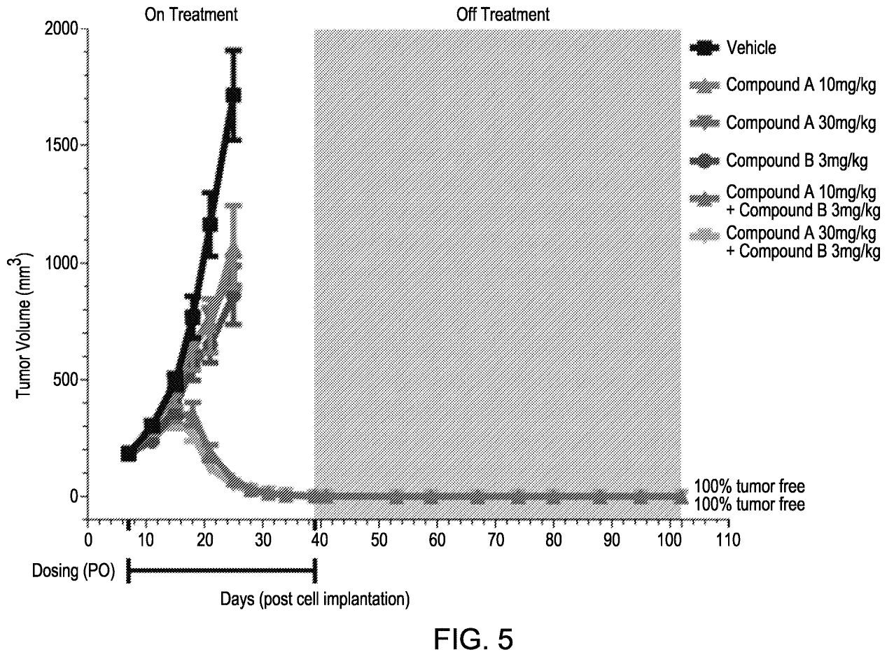

H838 cells (5×10⁶) were re-suspended in 100 μL of serum free media with 1:1 Matrigel and injected subcutaneously in the right flank of CB.17 SCID mice. Animals were sorted based on tumor volumes on day 18. Animals were orally dosed once daily for a total of 33 doses with either vehicle (0.1% Tween®80, 2% HPMC in DI water, pH 2), Compound A at 10 mg/kg or 30 mg/kg, Compound B at 3 mg/kg or a combination of Compound A and Compound B. Tumor volumes were measured twice a week with digital calipers. Plotted data represents group means±SEM for each group. N=10 for each group. Results show that a combination of Compound A and Compound B resulted in significant anti-tumor activity versus either single agent alone in H838 lung cancer xenografts (see FIG. 5).

What is claimed is:

1. A method of treating cancer in a patient in need thereof comprising administering a combination therapy of a therapeutically effective amount of Compound A or pharmaceutically acceptable salt thereof and a therapeutically effective amount of Compound B or pharmaceutically acceptable salt thereof to the patient.

2. The method of claim 1, wherein the cancer is a MTAP-null cancer.

3. The method of claim 1, wherein the cancer is an MTAP-deficient cancer, MTA-accumulating cancer, or a combination thereof.

4. The method of claim 1 or 2, wherein the cancer is a solid tumor.

5. The method of claim 4, wherein the tumor is malignant.

6. The method of any one of claims 1-4, wherein the cancer is lung cancer.

7. The method of any one of claims 1-4, wherein the cancer is pancreatic cancer.

8. The method of claim 2, wherein the MTAP-null cancer is lung cancer.

9. The method of claim 8, wherein the lung cancer is non-squamous cell lung cancer (NSCLC).

10. The method of claim 2, wherein the MTAP-null cancer is biliary tract cancer.

11. The method of claim 2, wherein the MTAP-null cancer is head and neck squamous cell carcinoma.

12. The method of claim 2, wherein the MTAP-null cancer is pancreatic adenocarcinoma.

13. The method of claim 2, wherein the MTAP-null cancer is gallbladder cancer.

14. The method of claim 2, wherein the MTAP-null cancer is mesothelioma.

15. The method of claim 2, wherein the cancer is not a primary brain tumor or lymphoma.

16. The method of any one of claims 1-15, wherein Compound A or salt thereof is administered orally.

17. The method of any one of claims 1-16 wherein Compound A or salt thereof is administered once daily.

18. The method of any one of claims 1-17, wherein Compound B or salt thereof is administered orally.

19. The method of any one of claims 1-18, wherein Compound A or salt thereof and Compound B or salt thereof are administered concurrently.

20. The method of any one of claims 1-18, wherein Compound A or salt thereof and Compound B or salt thereof are administered sequentially.

21. The method of any one of claims 1-20, wherein the therapeutically effective amount of Compound A or salt thereof is less than an amount of Compound A or salt thereof as a monotherapy.

22. The method of any one of claims 1-21, wherein the therapeutically effective amount of Compound B or salt thereof is less than an amount of Compound B or salt thereof as a monotherapy.

23. The method of any one of claims 1-22, wherein the combination therapy exhibits combination benefit compared to Compound A or salt thereof monotherapy.

24. The method of any one of claims 1-23, wherein the combination therapy exhibits combination benefit compared to Compound B or salt thereof monotherapy.

25. The method of any one of claims 1-24, wherein the cancer is deficient in CDKN2A.

26. A combination product comprising a therapeutically effective amount of Compound A or pharmaceutically acceptable salt thereof and a therapeutically effective amount of Compound B or pharmaceutically acceptable salt thereof.

27. Use of a therapeutically effective amount of Compound A or pharmaceutically acceptable salt thereof and a therapeutically effective amount of Compound B or pharmaceutically acceptable salt thereof in treating cancer.

28. Use of a therapeutically effective amount of Compound A or pharmaceutically acceptable salt thereof and a therapeutically effective amount of Compound B or pharmaceutically acceptable salt thereof in the manufacture of a medicament for treating cancer.

29. The use of claim 27 or 28, wherein the therapeutically effective amount of Compound A or pharmaceutically acceptable salt thereof and a therapeutically effective amount of Compound B or pharmaceutically acceptable salt thereof are administered concurrently.

30. The use of claim 27 or 28, wherein the therapeutically effective amount of Compound A or pharmaceutically acceptable salt thereof and a therapeutically effective amount of Compound B or pharmaceutically acceptable salt thereof are administered sequentially.
